(19) United States
(12) Patent Application Publication
(10) Pub. No.: US 2008/0206342 A1
(43) Pub. Date: Aug. 28, 2008

(54) Compositions and Methods For Increasing the Bioavailability of Pulmonarily Administered Insulin

(76) Inventor: Rosemary Kovelesky, San Francisco, CA (US)

Correspondence Address: NEKTAR THERAPEUTICS, 201 INDUSTRIAL ROAD, SAN CARLOS, CA, 94070, US

(21) Appl. No.: 11/795,056

(22) PCT Filed: Jan. 10, 2006
(86) PCT No.: PCT/US06/00641

§ 371 Date: Nov. 30, 2007

Related U.S. Application Data
(60) Provisional application No. 60/643,073, filed on Jan. 10, 2005.

(51) Int. Cl. A61K 9/14; A61K 38/28; A61P 3/10

(52) U.S. Cl. 424/489; 514/3

(57) ABSTRACT

A pharmaceutical composition suitable for pulmonary delivery includes insulin and EDTA. The presence of EDTA is effective to increase the relative pulmonary bioavailability of the insulin compared to the relative pulmonary bioavailability exhibited by a composition having the same components but absent the EDTA. The composition is in dry powder form. A method of treating or ameliorating diabetes or a related condition in a mammal includes administering by inhalation a pharmacologically effective amount of the composition. A method of improving the pulmonary bioavailability of an insulin composition includes adding EDTA to an insulin composition suitable for pulmonary delivery, wherein the composition is in dry powder form.

### FIELD OF THE INVENTION

This invention relates to pharmaceutical compositions of insulin with improved bioavailability and uses thereof.

### REFERENCES

U.S. Pat. No. 6,165,976.
U.S. Pat. No. 6,063,138.
U.S. Pat. No. 5,785,049.
U.S. Pat. No. 5,740,794.
U.S. Pat. No. 5,672,581.
U.S. Pat. No. 5,522,385.
U.S. Pat. No. 5,458,135.
U.S. Pat. No. 5,388,572.
U.S. Pat. No. 4,805,811.
U.S. Pat. No. 4,668,218.
U.S. Pat. No. 4,667,668.
WO 01/32144.
WO 99/16419.
WO 98/16205.
WO 97/41031.
WO 97/41833.
WO 96/32149.
WO 96/32096.
WO 95/09616.
European Patent No. EP472598 (1996).
European Patent No. EP 467172 (1994).
European Patent No. EP 129985 (1988).
Angell, C. A., “Formation of Glasses from Liquids and Biopolymers”, Science, 267, 1924-1935 (1995).
Gibbs and DiMarzio, “Nature of the Glass Transition and the Glassy State”, Journal of Chemical Physics, 28, 373-383 (1958).
Gonda, “Physico-chemical principles in aerosol delivery,” in Topics in Pharmaceutical Sciences 1991, Crommelin, D. J. and K. K. Midha, Eds., Medpharm Scientific Publishers, Stuttgart, pp. 95-117, 1992.
Hilsted, J., et al., Diabetologia 38, 680-684 (1995).
Masters, K., “Spray Drying Handbook”, 5th ed., John Wiley & Sons, Inc., NY, N.Y. (1991).
Patton, et al., Adv. Drug Delivery Reviews, 1, 35 (2-3), 235-247 (1999).
“Physician's Desk Reference”, 55th ed., Medical Economics, Montvale, N.J. (2001).
Remington: The Science & Practice of Pharmacy”, 19th ed., Williams & Williams, (1995).
Wolanczyk, J. P., “Differential Scanning Calorimetry Analysis of Glass Transitions”, Cryo-Letters, 10, 73-76 (1989).

All of the above publications, patents and patent applications are herein incorporated by reference in their entirety to the same extent as if the disclosure of each individual publication, patent application or patent was specifically and individually indicated to be incorporated by reference in its entirety.

### BACKGROUND OF THE INVENTION

Insulin is a protein hormone that is secreted by the β-cells of the islets of Langerhans in the pancreas. Insulin promotes the uptake of glucose by body cells, particularly in the liver and muscles, thereby controlling the concentration of glucose in the blood. The condition known as diabetes mellitus occurs in people who do not produce enough insulin or do not properly respond to insulin, resulting in the accumulation of large amounts of glucose in the blood and subsequent excretion in the urine.

In Type I diabetes, or insulin-dependent diabetes mellitus (IDDM), it is speculated that a virus or autoimmune reaction destroys the insulin-producing cells. Type I diabetes requires lifelong treatment, which means taking insulin several times daily. In type II diabetes, or non-insulin-dependent diabetes mellitus (NIDDM), the body still produces some insulin, but the quantity of insulin it produces is not sufficient, or the body does not respond to the hormone properly. Although type-II diabetes can frequently be managed through proper diet, exercise, and medication, insulin therapy is sometimes required.

Insulin therapy for type I and type II diabetes requires that acceptable blood glucose levels be maintained for extended periods of time. With careful monitoring of blood glucose levels followed by self-injections of insulin, diabetes can be successfully treated. In fact, it has been shown that more frequent monitoring accompanied by additional insulin significantly decreases some of the long-term effects of diabetes, such as eye, kidney and nerve problems. However, the inconvenience of frequent blood monitoring and the discomfort associated with self-administered injections contribute to poor patient compliance to prescribed therapy regimes. As a result, the search for alternative methods of delivery of insulin is ongoing.

Recently, delivery of therapeutics through pulmonary routes has proven to be advantageous for certain drugs. These devices eliminate the need for needles, limit irritation to the skin and body mucosa (common side effects of transdermally, iontophoretically, and intranasally delivered drugs), and eliminate the need for nasal and skin penetration enhancers (typical components of intranasal and transdermal systems that often cause skin irritations/dermatitis). Pulmonary administration is also economically attractive, amenable to patient self-administration, and is often preferred by patients over other alternative modes of administration. Liquid nebulizers, aerosol-based metered dose inhalers and dry powdered dispersion devices are some examples of pulmonary delivery devices. Dry powder inhalers are well-established as delivery devices for drugs such as bronchodilator and steroids in the treatment of respiratory tract diseases.

While pulmonary delivery of proteins and peptides, like insulin, has attracted much attention, formulation and aerosolization of these agents is a challenge due to their high molecular weight and low lipophilicity. Some of the problems unique to the development of inhaleable drug compositions are 1) the protein's tendency to become inactive in a dry-powder composition, 2) aggregation, 3) low flowability phenomena and 4) low bioavailability.

To date, no commercially viable inhaleable system for insulin has been developed (Patton, et al., Adv. Drug Delivery Reviews, 1, 35 (2-3), 235-247 (1999)), mainly due to low and variable bioavailability (Hilsted, J., et al., Diabetologia 38, 680-684 (1995)). A need still exists for compositions of insulin that provide bioavailability more closely resembling bioavailability of insulin delivered subcutaneously.

### SUMMARY OF THE INVENTION

The present invention provides a composition of insulin that yields improved bioavailability when delivered to the lung, by including ethylene diaminetetraacetic acid (EDTA). Less insulin is needed in the composition to achieve the same physiological effects, thereby significantly increasing the cost-effectiveness of insulin compositions. The pharmaceutical compositions of the present invention can be employed to treat or ameliorate diabetes and/or related conditions, and to provide improved bioavailability relative to insulin compositions without EDTA.

Accordingly, one aspect of the present invention provides a pharmaceutical composition suitable for pulmonary delivery comprising insulin and EDTA, wherein the presence of EDTA is effective to increase the relative pulmonary bioavailability of the insulin when compared to the relative pulmonary bioavailability of insulin provided by a composition having the same components but containing no EDTA. Preferably, the EDTA of the pharmaceutical composition is a sodium salt of ethylene diaminetetraacetic acid.

In a preferred embodiment, the EDTA comprises from about 5 to about 90 solid weight percent of the composition. The EDTA may comprise about 10 to about 85 solid weight percent, such as from about 30 to about 75 solid weight percent, or from about 50 to about 70 solid weight percent of the composition. The pharmaceutical composition of insulin and EDTA may be in dry powder form. Preferably, the particles of the powder may have a mass median diameter (MMD) from about 1-5 microns. In another preferred embodiment the particles of the powder may have a mass median aerodynamic diameter (MMAD) of less than about 10 microns, such as less than about 5 microns, or less than about 3.5 microns. The pharmaceutical composition may be a solution or suspension.

Another aspect of the present invention involves a method of treating or ameliorating diabetes or a related condition in a mammal. This method comprises administering by inhalation a pharmacologically effective amount of a pharmaceutical composition suitable for pulmonary delivery comprising insulin and EDTA. The EDTA in this composition is effective to increase the relative pulmonary bioavailability of the insulin when compared to the relative pulmonary bioavailability of insulin provided by a composition having the same components but containing no EDTA. When administered by inhalation, the pharmaceutical composition preferably results in a relative pulmonary bioavailability of insulin that is at least about 1.2 fold, such as at least about 1.5 fold, at least about 2 fold, or at least about 3 fold of the relative pulmonary bioavailability of insulin provided by a composition having the same components but containing no EDTA. In a preferred embodiment the insulin and EDTA composition is administered using a dry powder inhaler. In an alternate preferred embodiment the insulin and EDTA composition is administered using a nebulizer. In yet another preferred embodiment the insulin and EDTA composition is administered using a metered dose inhaler (MDI). The pharmacologically effective amount of insulin may be administered in a plurality of unit dosages.

Yet another embodiment involves a method of improving the pulmonary bioavailability of an insulin composition, comprising adding EDTA to an insulin composition suitable for pulmonary delivery.

### BRIEF DESCRIPTION OF THE DRAWINGS

FIG. 1 depicts the change of the C_{MAX} of insulin with increasing amounts of EDTA in the composition.

FIG. 2 illustrates the change of T_{MAX} of insulin with increasing amounts of EDTA in the composition.

FIG. 3 depicts the change of AUC (from 0 to infinity (inf.) hours after dosing) of insulin with increasing amounts of EDTA in the composition.

FIG. 4 shows the change of AUC (from 0 to 6 hours after dosing) of insulin with increasing amounts of EDTA in the composition.

FIG. 5 depicts the effect of EDTA on t_{1/2} of insulin.

FIG. 6 demonstrates the change of relative pulmonary bioavailability (RPB) of insulin with increasing amounts of EDTA in the composition.

### DETAILED DESCRIPTION OF THE INVENTION

### Definitions

The following terms as used herein have the meanings indicated.

“Insulin” as used herein includes proinsulin and encompasses any purified isolated polypeptide having part or all of the primary structural conformation (i.e., contiguous series of amino acid residues) and at least one of the biological properties of naturally occurring insulin. In general, the term “insulin” is meant to encompass natural and synthetically-derived insulin including glycoforms thereof as well as agonists and analogs thereof, including polypeptides having one or more amino acid modifications (deletions, insertions, or substitutions) to the extent that they substantially retain at least 80% or more of the therapeutic activity associated with full length insulin. The insulins of the present invention may be produced by any standard manner, including but not limited to pancreatic extraction, recombinant expression and in vitro polypeptide synthesis.

“Relative pulmonary bioavailability” is the percentage of an insulin-containing composition dose deposited in the lungs that is absorbed and enters the blood of a mammal relative to the percentage that is absorbed into the blood from an intramuscular or subcutaneous injection site. Representative model systems for determining relative pulmonary bioavailabilities include rat, dog, rabbit, and monkey. The compositions of the present invention are characterized by a relative pulmonary bioavailability of at least about 10% in plasma or blood, with relative pulmonary bioavailabilities generally ranging from about 10% to 60%, such as from about 20% to about 60%, or from about 40% to about 60%. Generally, depending upon the EDTA content, a composition of the present invention will possess a relative pulmonary bioavailability of at least about 10%, such as at least about 15%, at least about 20%, at least about 30%, at least about 40%, at least about 50%, or at least about 60% or greater. Relative pulmonary bioavailability may be ascertained by measuring absorption following direct intratracheal administration, pulmonary instillation, or inhalation.

Unless otherwise noted, relative pulmonary bioavailability is determined under the following conditions. Female beagle dogs are exposed to inhaled powder for 15 minutes at a target aerosol concentration of 80 μg/L. These animals are dosed with 50 μg/kg via subcutaneous administration approximately 1-week after inhalation exposures. Tidal volume, breathing rate, and minute volume are monitored prior to and throughout the exposure period. Blood is collected for analysis of plasma levels of insulin at various time points following inhalation and subcutaneous administration.

The female beagle dogs are acclimated to the restraint slings prior to beginning the study. The weight range of the animals at the start of the study may range from 8 to 11 kg. The age of the animals at the start of the study may range from 33 to 37 weeks.

Animals are pair housed in stainless steel cages except on the days of exposure. Each animal is individually housed on the day of exposure in order to monitor their feeding regimen. Rooms are thermostatically set to maintain a temperature of 70° F. and maintain the actual temperature within ±8° F. from that set point. The environmental control system is designed to maintain a relative humidity of 20% and a maximum of 80%. Light is on a 12-hour cycle, with lights on between 0600 and 1800 hours. Subsequently, lights are off between 1800 and 0600 hours except when blood samples are collected. Animals are fed once daily with Hill's Science Diet. Animals are fasted for approximately 12 hours prior to exposure. Tap water is provided ad libitum except during exposures.

All dogs are exposed for 15 minutes to aerosolized powder. The targeted deposited lung dose is 100 Ag/kg of body weight. Approximately 7 days after pulmonary administration of powder, all dogs are dosed with 50 μg/kg insulin of body weight via subcutaneous administration.

The dogs are tested while standing in restraint slings. Two layers of 0.03 inch latex sheets are placed around the animals' necks to form a nonrestrictive airtight seal. A custom built, 11-L head-dome, similar to that described by Allen et al. (J Appl Toxicol 1995; 15:13-17) is placed over the dogs' heads and secured to the sling. Airflow is exhaust driven via a transvector located on the exhaust side of the dome. Because the helmet is airtight and the neck is sealed, this constituted a head-only exposure system. The total flow rate through the dome is approximately 7.5 L/min. To the inlet is connected a Pulmonary Delivery System (PDS), as disclosed in U.S. Pat. No. 6,257,233, which is incorporated by reference herein in its entirety. The PDS aerosolizes the powder.

The dose of insulin inhaled during a 15-minute exposure is estimated as follows: the mean minute volume (mL) during the 15 minute exposure is multiplied by the exposure duration to yield the total air breathed (L) during the inhalation exposure. This value is multiplied by the aerosol concentration (μg/L) to determine total dose (mg). The inhaled dose (μg/kg) is calculated by dividing the total dose (μg) by the animal's body weight (kg).

The dose of insulin deposited in the lungs is estimated as follows: inhaled dose (μg/kg) is multiplied by 20 percent to yield estimated deposited lung dose (μg/kg). Aerosols with a MMAD ranging from 1-2 μm MMAD have been shown to deposit in the lung with approximately a 20 percent efficiency (Schlesinger R B. 1985. Comparative deposition of inhaled aerosols in experimental animals and humans: a review. J Toxicol Environ Health 15:197-214).

All animals are weighed on Days −5, 0, and 7. Breathing patterns (tidal volume, breathing frequency, and minute volume) are monitored using a size ‘0’ pneumotachograph connected to a port on the head-dome. The signals are collected on a personal computer using the Buxco XA Data Acquisition System (Buxco Electronics, Inc, Sharon, Conn.). At least 15 minutes of preexposure data are collected before the exposures are begun, followed by data collection throughout the 15-minute exposure period. All data is analyzed as 5-minute averages.

Approximately 2 to 3 mL of blood is collected in EDTA vacutainer tubes from the cephalic or jugular vein prior to exposure and at 0.25, 0.5, 1, 2, 3, 4, 6, 8, and 12 hours postexposure. To obtain the plasma, each tube is centrifuged at approximately 3000 rpm for 10 minutes at 10° C. The plasma samples are stored at −70° C. until assayed. The bioavailability is then calculated.

The “solid weight percentage” of an ingredient in a composition refers to the weight/weight percentage of the ingredient in the composition when the weight of water is excluded. Thus, the solid weight percentage of an ingredient is calculated by dividing the dry weight of the ingredient with the total dry weight of the composition, then multiplying by 100.

“Distribution phase”, in reference to the half-life of insulin, refers to the initial rapid phase during which insulin disappears from the plasma. The “terminal slow” or “elimination phase”, in reference to the half-life of insulin, refers to the terminal slow phase during which insulin is eliminated from the body.

“EDTA” refers to ethylene diaminetetraacetic acid and any salts thereof. The salts are preferably metal salts, such as alkaline or alkaline earth salts, e.g., sodium salts of ethylene diaminetetraacetic acid. The EDTA of the present invention preferably contains no calcium.

A composition that is “suitable for pulmonary delivery” refers to a composition that is capable of being aerosolized and inhaled by a subject so that a portion of the aerosolized particles reaches the lungs to permit penetration into the alveoli. Such a composition is considered to be “respirable” or “inhaleable”.

“Aerosolized” particles are liquid or solid particles that are suspended in a gas (typically air), typically as a result of actuation (or firing) of an inhalation device such as a dry powder inhaler, an atomizer, a metered dose inhaler, or a nebulizer.

A “nebulizer” is a device that forces compressed air through a solution of a drug so that a fine spray can be delivered to a facemask and inhaled. Nebulizers are often used to administer drugs to those who lack the ability to use a metered-dose or breath-activated inhaler.

A composition in “dry powder form” is a powder composition that typically contains less than about 10% moisture.

“Mass median diameter”, or “MMD”, is a measure of mean particle size, since the powders of the present invention are generally polydisperse (i.e., consist of a range of particle sizes). MMD values can be determined by centrifugal sedimentation, or any other commonly employed technique for measuring mean particle size (e.g., electron microscopy, light scattering, laser diffraction).

“Mass median aerodynamic diameter”, or “MMAD”, is a measure of the aerodynamic size of a dispersed particle. The aerodynamic diameter is used to describe an aerosolized powder in terms of its settling behavior, and is the diameter of a unit density sphere having the same settling velocity, in air, as the particle. The aerodynamic diameter encompasses particle shape, density and physical size of a particle. As used herein, MMAD refers to the midpoint or median of the aerodynamic particle size distribution of an aerosolized powder determined by cascade impaction, unless otherwise indicated.

A “dry powder inhaler” is a device that is loaded with capsules of the drug in powder form. Generally, the inhaler is activated by taking a breath; the capsule is punctured and the powder is dispersed so that it can be inhaled in “Spinhalers” or “Rotahalers”. “Turbohalers” are fitted with canisters that deliver measured doses of the drug in powder form.

A “metered dose inhaler” or “MDI” is a device that delivers a measured dose of a drug in the form of a suspension of extremely small liquid or solid particles, which is dispensed from the inhaler by a propellant under pressure. Such inhalers are placed into the mouth and depressed (activated) to release the drug as the individual takes a breath.

“Emitted Dose” or “ED” provides an indication of the delivery of a drug composition from a suitable inhaler device after a firing or dispersion event. More specifically, for dry powder compositions, the ED is a measure of the percentage of powder which is drawn out of a unit dose package and which exits the mouthpiece of an inhaler device. The ED is defined as the ratio of the dose delivered by an inhaler device to the nominal dose (i.e., the mass of powder per unit dose placed into a suitable inhaler device prior to firing). The ED is an experimentally-determined parameter, and is typically determined using an in-vitro device set up which mimics patient dosing. To determine an ED value, a nominal dose of dry powder, typically in unit dose form, is placed into a suitable dry powder inhaler (such as that described in U.S. Pat. No. 5,785,049, assigned to Inhale Therapeutic Systems) that is then actuated, dispersing the powder. The resulting aerosol cloud is then drawn by vacuum from the device, where it is captured on a tared filter attached to the device mouthpiece. The amount of powder that reaches the filter constitutes the emitted dose. For example, for a 5 mg dry powder-containing dosage form placed into an inhalation device, if dispersion of the powder results in the recovery of 4 mg of powder on a tared filter as described above, then the emitted dose for the dry powder composition is: 4 mg (delivered dose)/5 mg (nominal dose)×100=80%. For non-homogenous powders, ED values are based on the amount of drug rather than on the total powder weight. They provide an indication of the amount of drug delivered from an inhaler device after firing rather than the amount of dry powder delivered. Similarly for MDI and nebulizer dosage forms, the ED corresponds to the percentage of drug which is drawn from a dosage form and which exits the mouthpiece of an inhaler device.

A “solution” of the present invention is a composition in which the insulin is dissolved.

A “suspension” of the present invention is a composition in which the insulin exists as insoluble particles distributed in a fluid (liquid or gas).

A “pharmaceutically acceptable excipient or carrier” refers to an excipient that may optionally be included in the compositions of the present invention. Preferred are excipients that can be taken into the lungs with no significant adverse toxicological effects to the subject, particularly to the lungs of the subject.

The “glass transition temperature (Tg)”, as used herein, is the onset of a temperature range at which a composition changes from a glassy or vitreous state to a syrup or rubbery state. Generally, Tg is determined using differential scanning calorimietry (DSC). The standard designation for Tg is the temperature at which onset of the change of heat capacity (Cp) of the composition occurs upon scanning through the transition. The definition of Tg, however, can be arbitrarily defined as the onset, midpoint or endpoint of the transition. For purposes of the present invention, we will use the onset of the changes in Cp when using DSC and DER. See the article entitled “Formation of Glasses from Liquids and Biopolymers” by C. A. Angell: Science, 267, 1924-1935 (Mar. 31, 1995) and the article entitled “Differential Scanning Calorimetry Analysis of Glass Transitions” by Jan P. Wolanczyk: Cryo-Letters, 10, 73-76 (1989). For detailed mathematical treatment see “Nature of the Glass Transition and the Glassy State” by Gibbs and DiMarzio: Journal of Chemical Physics, 28, NO. 3, 373-383 (March, 1958). These articles are incorporated herein by reference.

A “pharmacologically effective amount” or “physiologically effective amount” is the amount of an insulin present in a therapeutic composition as described herein that is needed to provide a desired level of insulin in the bloodstream to result in a target blood glucose level. The precise amount will depend upon numerous factors, e.g., the delivery device employed, the components and physical characteristics of the therapeutic composition, intended patient population, and the like, and can readily be determined by one skilled in the art, based upon the information provided herein.

“Treating or ameliorating” a disease or medical condition means reducing or eliminating the symptoms of the disease or medical condition.

The term “diabetes and related conditions” refers to diseases or medical conditions caused by the lack or inaction of insulin. Diabetes and related conditions include type I and type II diabetes, particularly type I diabetes.

### Insulin and EDTA

The composition of the present invention comprises insulin and EDTA, wherein EDTA increases the bioavailability of insulin when the composition is delivered to the lung, particularly by inhalation. As demonstrated in Example 1, addition of EDTA to an insulin-containing composition led to significant increases in the maximum plasma concentration (C_{MAX}), time to maximum plasma concentration (T_{MAX}), area under the plasma concentration vs. time curve (AUC), and the relative pulmonary bioavailability of insulin. Thus, EDTA is capable of enhancing absorption of insulin by the lung, which leads to a higher blood insulin level.

It is surprising that EDTA enhances bioavailability of insulin. It was previously taught that EDTA does not enhance absorption of insulin in the lung (U.S. Pat. No. 6,165,976). In contrast, our results clearly indicate that EDTA promotes absorption of insulin delivered to the lung. Therefore, the present invention provides compositions that can be used to increase the bioavailability of pulmonarily delivered insulin, thereby enhancing the cost-effectiveness of insulin compositions since less insulin needs to be included. The present invention further provides methods for treating or ameliorating diabetes or related conditions, as well as methods for improving bioavailability of insulin, by using the compositions of the present invention.

The compositions of the present invention typically comprise EDTA at a solid weight percentage of about 0.1% to about 95%, such as about 10% to about 85%, about 30% to about 75%, or about 50% to about 70%. Depending upon the amount of excipient, a composition of the present invention will comprise EDTA at a solid weight percentage of at least about one of the following: 10%, 15%, 20%, 30%, 40%, 50%, 60%, 70%, 80%, 90% or greater. The EDTA may be ethylene diaminetetraacetic acid or any salt thereof. The salt is preferably a metal salt, such as an alkaline or alkaline earth salt, e.g., a sodium salt such as Na₂EDTA or Na₄EDTA.

Compositions and corresponding doses of insulin vary with the bioactivity of the insulin employed. For example, injectable insulin is measured in USP Insulin Units; one unit (U) of insulin is equal to the amount required to reduce the concentration of blood glucose in a fasting rabbit to 0.45 mg/ml (2.5 mM). Typical concentrations of insulin preparations for injection range from 30-100 Units/mL, which is about 3.6 mg of insulin per mL. The amount of insulin required to achieve the desired physiological effect in a patient will vary not only with the particulars of the patient and his disease (e.g., type I vs. type II diabetes) but also with the strength and particular type of insulin used. For instance, dosage ranges for regular insulin (rapid acting) are from about 0.3 to 2 U insulin per kilogram of body weight per day. The compositions of the present invention are, in one aspect, effective to achieve in patients undergoing therapy, a fasting blood glucose concentration between about 90 and 140 mg/dl and a postprandial value below about 250 mg/dl. Precise dosages can be determined by one skilled in the art when coupled with the pharmacodynamics and pharmacokinetics of the precise insulin-EDTA composition employed for a particular route of administration, and can readily be adjusted in response to periodic glucose monitoring.

Individual dosages (on a per inhalation basis) for inhaleable insulin compositions are typically in the range of from about 0.5 mg to 15 mg insulin, where the desired overall dosage is typically achieved in about 1-10 breaths, such as in about 1 to 4 breaths. On average, the overall dose of insulin administered by inhalation per dosing session will range from about 10 U to about 400 U, with each individual dosage or unit dosage form (corresponding to a single inhalation) containing from about 5 U to 400 U.

As stated above, one preferred route of administration for the insulin-EDTA composition of the present invention is by inhalation to the lung. The amount of insulin in the composition will be that amount necessary to deliver a therapeutically effective amount of insulin per unit dose to achieve at least one of the therapeutic effects of native insulin, i.e., the ability to control blood glucose levels to near normoglycemia. In practice, this will vary widely depending upon the particular insulin, its activity, the severity of the diabetic condition to be treated, the patient population, the stability of the composition, and the like. The composition will generally contain, in terms of solid weight, anywhere from about 1% to about 99% insulin, such as from about 2% to about 95%, or from about 5% to about 85%. The percentage of insulin in the composition will also depend upon the relative amounts of excipients/additives contained in the composition. More specifically, the composition will typically contain at least about one of the following solid weight percentages of insulin: 10%, 20%, 30%, 40%, 50%, 60%, 70%, 80%, 90% or more. Preferably, powder compositions will contain at least about 60%, e.g., about 60-100% by weight insulin. It is to be understood that more than one insulin may be incorporated into the compositions described herein and that the use of the term “insulin” in no way excludes the use of two or more insulins or a combination of insulin with another active agent.

The molar ratio of EDTA to insulin in the compositions of the present invention may range from about 0.5 to about 2000. The ratio may be from about 100 to about 1000, such as from about 200 to about 500. The optimal molar ratio of EDTA to insulin may be determined by a person of ordinary skill in the art, and will generally be about one of the following: 0.5, 1, 2, 5, 10, 20, 50, 100, 200, 300, 400, 500, 600, 700, 800, 900, 1000 or greater.

The effect of a composition comprising insulin and EDTA in improving bioavailability of insulin can be determined by any method known in the art. By way of example, the amount of plasma insulin may be measured immunologically (e.g., using an enzyme-linked immunosorbent assay and specific antibodies against insulin), biochemically (e.g., using mass spectrometry) or functionally (e.g., by measuring blood glucose level). The composition of the present invention typically exhibits an improved relative pulmonary bioavailability, which is the percentage of an insulin-containing composition dose deposited in the lungs that is absorbed and enters the blood of a mammal relative to the percentage that is absorbed into the blood from an intramuscular or subcutaneous injection. For example, if the bioavailability of a subcutaneous insulin composition is 60% F, and that of a pulmonary insulin composition is 12% F, the relative pulmonary bioavailability of the pulmonary composition is 20%.

Generally, the relative pulmonary bioavailability of a composition of the present invention is at least about 1.1 fold as much as the relative pulmonary bioavailability of a composition absent EDTA. For example, if a composition lacking EDTA yields a relative pulmonary bioavailability of 20%, the same composition containing EDTA would typically have an improved relative pulmonary bioavailability that is at least 22% (1.1 fold of 20%). The relative pulmonary bioavailability of the composition of the present invention may be at least about 1.2 fold, such as at least about 1.5 fold, at least about 2 fold, or at least about 3 fold, as much as the relative pulmonary bioavailability of a comparable composition that lacks EDTA.

The compositions of the present invention also result in improved pharmacokinetic features, such as C_{MAX}, T_{MAX}, and AUC. An EDTA-containing composition of the present invention typically displays a C_{MAX}, T_{MAX} or AUC that is at least about 1.1 fold, such as at least about 1.2 fold, at least about 1.5 fold, at least about 2 fold, or at least about 3 fold, when compared to the corresponding value of a composition lacking EDTA but is otherwise the same as the composition of the present invention.

### Excipients

The composition may further comprise excipients, solvents, stabilizers, etc., depending upon the particular mode of administration and dosage form. Preferred are carbohydrate excipients, either alone or in combination with other excipients or additives. Representative carbohydrates for use in the compositions of the present invention include sugars, derivatived sugars such as alditols, aldonic acids, esterified sugars, and sugar polymers. Exemplary carbohydrate excipients suitable for use in the present invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), pyranosyl sorbitol, myoinositol and the like. Preferred are non-reducing sugars, sugars that can form a substantially dry amorphous or glassy phase when combined with an insulin, and sugars possessing relatively high glass transition temperatures, or Tgs (e.g., Tgs greater than 40° C., such as greater than 50° C., greater than 60° C., greater than 70° C., or having Tgs of 80° C. and above).

Additional excipients include amino acids, peptides and particularly oligomers comprising 2-9 amino acids, such as 2-5 mers, and polypeptides, all of which may be homo or hetero species. Representative amino acids include glycine (gly), alanine (ala), valine (val), leucine (leu), isoleucine (ile), methionine (met), proline (pro), phenylalanine (phe), trytophan (trp), serine (ser), threonine (thr), cysteine (cys), tyrosine (tyr), asparagine (asp), glutamic acid (glu), lysine (lys), arginine (arg), histidine (his), norleucine (nor), and modified forms thereof. One preferred amino acid is leucine.

Also preferred for use as excipients in inhaleable compositions are di- and tripeptides containing two or more leucyl residues, as described in WO 01/32144, incorporated herein by reference in its entirety.

Also preferred are di- and tripeptides having a glass transition temperature greater than about 40° C., such as greater than 50° C., greater than 60° C., or greater than 70° C.

Although less preferred due to their limited solubility in water, additional stability and aerosol performance-enhancing peptides for use in the present invention are 4-mers and 5-mers containing any combination of amino acids as described above. More preferably, the 4-mer or 5-mer will comprise two or more leucine residues. The leucine residues may occupy any position within the peptide, while the remaining (i.e., non-leucyl) amino acids positions are occupied by any amino acid as described above, provided that the resulting 4-mer or 5-mer has a solubility in water of at least about 1 mg/ml. Preferably, the non-leucyl amino acids in a 4-mer or 5-mer are hydrophilic amino acids such as lysine, to thereby increase the solubility of the peptide in water.

Polyamino acids, and in particular, those comprising any of the herein described amino acids, are also suitable for use as stabilizers. Preferred are polyamino acids such as poly-lysine, poly-glutamic acid, and poly(lys, ala).

Additional excipients and additives useful in the present compositions and methods include but are not limited to proteins, non-biological polymers, and biological polymers, which may be present singly or in combination. Suitable excipients are those provided in WO 96/32096 and WO 98/16205. Preferred are excipients having glass transition temperatures above about 35° C., such as above about 40° C., above 45° C., or above about 55° C.

Exemplary protein excipients include albumins such as human serum albumin (HSA), recombinant human albumin (rHA), gelatin, casein, hemoglobin, and the like. The compositions may also include a buffer or a pH-adjusting agent, typically but not necessarily a salt prepared from an organic acid or base. Representative buffers include organic acid salts of citric acid, ascorbic acid, gluconic acid, carbonic acid, tartaric acid, succinic acid, acetic acid, or phthalic acid. Other suitable buffers include Tris, tromethamine hydrochloride, borate, glycerol phosphate and phosphate. Amino acids such as glycine are also suitable.

The compositions of the present invention may also include polymeric excipients/additives, e.g., polyvinylpyrrolidones, derivatized celluloses such as hydroxymethylcellulose, hydroxyethylcellulose, and hydroxypropylmethylcellulose, Ficolls (a polymeric sugar), hydroxyethylstarch (BES), dextrates (e.g., cyclodextrins, such as 2-hydroxypropyl-β-cyclodextrin and sulfobutylether-β-cyclodextrin), polyethylene glycols, and pectin.

The compositions may further include flavoring agents, taste-masking agents, inorganic salts (e.g., sodium chloride), antimicrobial agents (e.g., benzalkonium chloride), sweeteners, antioxidants, antistatic agents, surfactants (e.g., polysorbates such as “TWEEN 20” and “TWEEN 80”, and pluronics such as F68 and F88, available from BASF), sorbitan esters, lipids (e.g., phospholipids such as lecithin and other phosphatidylcholines, phosphatidylethanolamines, although preferably not in liposomal form), fatty acids and fatty esters, steroids (e.g., cholesterol), and chelating agents (e.g., zinc and other such suitable cations). The use of certain di-substituted phosphatidylcholines for producing perforated microstructures (i.e., hollow, porous microspheres) is described in greater detail below. Other pharmaceutical excipients and/or additives suitable for use in the compositions according to the present invention are listed in “Remington: The Science & Practice of Pharmacy”, 19^{th} ed., Williams & Williams, (1995), and in the “Physician's Desk Reference”, 55th ed., Medical Economics, Montvale, N.J. (2001).

Preferred compositions in accordance with the present invention are absent penetration enhancers, which can cause irritation and are toxic at the high levels often necessary to provide substantial enhancement of absorption. Specific enhancers, which are typically absent from the compositions of the present invention, are the detergent-like enhancers such as deoxycholate, laureth-9, DDPC, glycocholate, and the fusidates. Certain enhancers, however, such as those that protect insulin from enzyme degradation, e.g., protease and peptidase inhibitors such as alpha-1 antiprotease, captropril, thiorphan, and the HIV protease inhibitors, may, in certain embodiments of the present invention, be incorporated in the composition of the present invention. In yet another embodiment, the composition of the present invention is preferably absent liposomes, lipid matrices, and encapsulating agents.

Generally, the pharmaceutical compositions of the present invention will contain from about 1% to about 99% by weight excipient, such as from about 5%-98% by weight excipient, or from about 15-95% by weight excipient. The spray-dried compositions may contain from about 0-50% by weight excipient, such as from 0-40% by weight excipient. In general, a high insulin concentration is desired in the final pharmaceutical composition. Typically, the optimal amount of excipient/additive is determined experimentally, i.e., by preparing compositions containing varying amounts of excipients (ranging from low to high), examining the chemical and physical stability of insulin, MMADs and dispersibilities of the pharmaceutical compositions, and then further exploring the range at which optimal aerosol performance is attained with no significant adverse effect upon insulin stability.

### Preparing Dry Powders

Dry powder compositions of the present invention comprising insulin and EDTA may be prepared by any of a number of drying techniques, such as spray drying. Spray drying of the compositions is carried out, for example, as described generally in the “Spray Drying Handbook”, 5^{th} ed., K. Masters, John Wiley & Sons, Inc., NY, N.Y. (1991), and in WO 97/41833 (1997) and WO 96/32149 (1996), the contents of which are incorporated herein by reference.

Solutions comprising insulin and EDTA are spray-dried in a conventional spray drier, such as those available from commercial suppliers such as Niro A/S (Denmark), Buchi (Switzerland) and the like, resulting in a dispersible, dry powder. Optimal conditions for spray drying the insulin-EDTA solutions will vary depending upon the composition components, and are generally determined experimentally. The gas used to spray dry the material is typically air, although inert gases such as nitrogen or argon are also suitable. Moreover, the temperature of both the inlet and outlet of the gas used to dry the sprayed material is such that it does not cause degradation of insulin in the sprayed material. Such temperatures are typically determined experimentally, although generally, the inlet temperature will range from about 50° C. to about 200° C., while the outlet temperature will range from about 30° C. to about 150° C. Preferred parameters include atomization pressures ranging from about 20-150 psi, such as from about 30-40 to 100 psi. Typically, the atomization pressure employed will be one of the following (psi): 20, 30, 40, 50, 60, 70, 80, 90, 100, 110, 120 or above.

Respirable compositions of the present invention having the features described herein may also be produced by drying certain composition components which result in formation of a perforated microstructure powder as described in WO 99/16419, the entire contents of which are incorporated by reference herein. The perforated microstructure powders typically comprise spray-dried, hollow microspheres having a relatively thin porous wall defining a large internal void. The perforated microstructure powders may be dispersed in a selected suspension media (such as a non-aqueous and/or fluorinated blowing agent) to provide stabilized dispersions prior to drying. The use of relatively low density perforated (or porous) microstructures or microparticulates significantly reduces attractive forces between the particles, thereby lowering the shear forces, increasing the flowability and dispersibility of the resulting powders, and reducing the degradation by flocculation, sedimentation or creaming of the stabilized dispersions thereof.

Less preferably, powders may be prepared by lyophilization, vacuum drying, spray freeze drying, super critical fluid processing (e.g., as described in Hanna, et al., U.S. Pat. No. 6,063,138), air drying, or other forms of evaporative drying.

In yet another approach, dry powders may be prepared by agglomerating the powder components, sieving the materials to obtain agglomerates, spheronizing to provide a more spherical agglomerate, and sizing to obtain a uniformly sized product, as described in WO 95/09616, 1995, incorporated herein by reference.

Dry powders may also be prepared by blending, grinding, sieving or jet milling composition components in dry powder form.

Once formed, the dry powder compositions may be maintained under dry (i.e., relatively low humidity) conditions during manufacture, processing, and storage. Irrespective of the drying process employed, the process will preferably result in inhaleable, highly dispersible particles comprising insulin and EDTA.

### Features of Dry Powder Compositions

Preferred powders of the present invention are further characterized by several features, most notably, (i) consistently high dispersibilities, which are maintained, even upon storage, (ii) small aerodynamic particles sizes (MMADs), (iii) improved fine particle dose values, i.e., powders having particles sized less than 5 microns MMAD, all of which contribute to the improved ability of the powder to penetrate to the tissues of the lower respiratory tract (i.e., the alveoli) for delivery to the systemic circulation. These physical characteristics of the inhaleable powders of the present invention, to be described more fully below, are important in maximizing the efficiency of aerosolized delivery of such powders to the deep lung.

Dry powders of the present invention are composed of aerosolizable particles effective to penetrate into the lungs. The particles of the present invention may have a mass median diameter (MMD) of less than about 20 μm, such as less than about 10 am, less than about 7.5 μm, less than about 4 μm, or less than about 3.5 μm, and usually are in the range of 0.1 μm to 5 μm in diameter. Preferred powders are composed of particles having an MMD from about 1 to 5 μm. In some cases, the powder will also contain non-respirable carrier particles such as lactose, where the non-respirable particles are typically greater than about 40 microns in size.

The preferred powders of the present invention may also be characterized by an aerosol particle size distribution less than about 10 μm mass median aerodynamic diameter (MMAD), such as less than about 5 μm, less than 4.0 μm, less than 3.5 μm, or less than 3 μm. The mass median aerodynamic diameters of the powders will characteristically range from about 0.1-10 μm, such as from about 0.2-5.0 μm MMAD, from about 1.0-4.0 μm MMAD, or from about 1.5 to 3.0 μm. Small aerodynamic diameters are generally achieved by a combination of optimized spray drying conditions and choice and concentration of excipients.

The powders of the present invention may also be characterized by their densities. The powder will generally possess a bulk density from about 0.1 to 10 g/cubic centimeter, such as from about 0.1-2 g/cubic centimeter, or from about 0.15-1.5 g/cubic centimeter. In one embodiment of the present invention, the powders have big and fluffy particles with a density of less than about 0.4 g/cubic centimeter and an MMD between 5 and 30 microns. It is worth noting that the relationship of diameter, density and aerodynamic diameter can be determined by the following formula (Gonda, 1992):

aerodynamic diameter=diameter\sqrt{density}.

The powders will generally have a moisture content below about 20% by weight, such as below about 10% by weight, or below about 5% by weight. Such low moisture-containing solids tend to exhibit a greater stability upon packaging and storage.

Additionally, the spray drying methods and stabilizers described herein are effective to provide highly dispersible insulin-EDTA compositions. Generally, the emitted dose (ED) of these powders is greater than 30%, such as greater than 40%, greater than 50%, or greater than 60%.

The compositions described herein also typically possess good stability with respect to both chemical stability and physical stability, i.e., aerosol performance over time. Generally, with respect to chemical stability, the insulin contained in the composition will degrade by no more than about 10% upon spray drying. That is to say, the powder will possess at least about 90% intact insulin, such as at least about 95% intact insulin, or at least about 97% or greater intact insulin. Preferably, the spray drying process will result in powders having less than about 10% total protein aggregates, that is to say, greater than 90% by weight of the insulin being in monomeric form.

With respect to aerosol performance, compositions of the present invention are generally characterized by a drop in emitted dose of no more than about 20%, such as no more than about 15%, or no more than about 10%, when stored under ambient conditions for a period of three months.

### Administration of the Composition

The compositions as described herein may be delivered using any suitable dry powder inhaler (DPI), i.e., an inhaler device that utilizes the patient's inhaled breath as a vehicle to transport the dry powder drug to the lungs. Preferred are dry powder inhalation devices as described in Patton, J. S., et al., U.S. Pat. No. 5,458,135, Oct. 17, 1995; Smith, A. E., et al., U.S. Pat. No. 5,740,794, Apr. 21, 1998; and in Smith, A. E., et. al., U.S. Pat. No. 5,785,049, Jul. 28, 1998, herein incorporated by reference. When administered using a device of this type, the powdered medicament is contained in a receptacle having a puncturable lid or other access surface, preferably a blister package or cartridge, where the receptacle may contain a single dosage unit or multiple dosage units. Convenient methods for filling large numbers of cavities (i.e., unit dose packages) with metered doses of dry powder medicament are described, e.g., in Parks, D. J., et al., WO 97/41031, Nov. 6, 1997, incorporated herein by reference.

Other dry powder dispersion devices for pulmonary administration of dry powders include those described, for example, in Newell, R. E., et al, European Patent No. EP 129985, Sep. 7, 1988; in Hodson, P. D., et al., European Patent No. EP472598, Jul. 3, 1996; in Cocozza, S., et al., European Patent No. EP 467172, Apr. 6, 1994, and in Lloyd, L. J. et al., U.S. Pat. No. 5,522,385, Jun. 4, 1996, incorporated herein by reference. Also suitable for delivering the dry powders of the present invention are inhalation devices such as the Astra-Draco “TURBUHALER”. This type of device is described in detail in Virtanen, R., U.S. Pat. No. 4,668,218, May 26, 1987; in Wetterlin, K., et al., U.S. Pat. No. 4,667,668, May 26, 1987; and in Wetterlin, K., et al., U.S. Pat. No. 4,805,811, Feb. 21, 1989, all of which are incorporated herein by reference. Other suitable devices include dry powder inhalers such as Rotahaler® (Glaxo), Discus® (Glaxo), Spiros™ inhaler (Dura Pharmaceuticals), and the Spinhaler® (Fisons). Also suitable are devices which employ the use of a piston to provide air for either entraining powdered medicament, lifting medicament from a carrier screen by passing air through the screen, or mixing air with powder medicament in a mixing chamber with subsequent introduction of the powder to the patient through the mouthpiece of the device, such as described in Mulhauser, P., et al, U.S. Pat. No. 5,388,572, Sep. 30, 1997, incorporated herein by reference.

The compositions of the present invention may also be delivered using a pressurized, metered dose inhaler (MDI), e.g., the Ventolin® metered dose inhaler, containing a solution or suspension of drug in a pharmaceutically inert liquid propellant, e.g., a chlorofluorocarbon or fluorocarbon, as described in Laube, et al., U.S. Pat. No. 5,320,094, Jun. 14, 1994, and in Rubsamen, R. M., et al, U.S. Pat. No. 5,672,581 (1994), both incorporated herein by reference.

Alternatively, the compositions described herein may be dissolved or suspended in a solvent, e.g., water or saline, and administered by nebulization. Nebulizers for delivering an aerosolized solution include the AERx™ (Aradigm), the Ultravent® (Mallinkrodt), the Pari LC Plus™ or the Pari LC Star™ (Pari GmbH, Germany), the DeVilbiss Pulmo-Aide, and the Acorn II® (Marquest Medical Products).

The following examples are offered to illustrate this invention and are not to be construed in any way as limiting the scope of the present invention.

### EXAMPLES

In the examples below, the following abbreviations have the following meanings. Abbreviations not defined have their generally accepted meanings.

MATERIALS AND METHODS

| | |
|---|---|
| ° C. = | degree Celsius |
| hr = | hour |
| min = | minute |
| sec = | second |
| μM = | micromolar |
| mM = | millimolar |
| M = | molar |
| ml = | milliliter |
| μl = | microliter |
| mg = | milligram |
| μg = | microgram |
| PBS = | phosphate buffered saline |
| RPB = | relative pulmonary bioavailability |

### Test System and Animal Husbandry

Pre-cannulated (jugular vein) Sprague Dawley (JVC) rats (250-300 grams), with an access port threaded under the skin externalizing at the nape of the neck, were supplied by Hilltop Lab Animals Inc. (P.O. Box 183, Scottdale, Pa. 15683). The jugular cannula of each was filled with a solution (lumen filler) of pharmaceutical grade Polyvinyl-Pyrrolidone (PVP-MW 40,000), physiological saline, and sodium heparin to retain potency. The nylon filament plug sealing the cannula was removed and replaced with a blunt needle cannula (230×1″, Monoject, VWR #53498-484). A physical exam by a veterinary technician was done to ascertain the good health of the rats. Until the study began, the rats were housed in individual stainless steel cages.

### Materials and Reagents

The insulin used for these examples was a 60% insulin dry powder composition (INHALE lot number B1887) stored at −20° C. However, any biologically active insulin may be used. The EDTA in these examples was a disodium salt, dihydrate, crystal of ethylene diaminetetraacetic acid obtained from JT. Baker, (lot number K34607). The PBS was used as supplied by the manufacturer (any manufacturer can be used).

The insulin stock solutions (MNH-192, Lot# B1887) were 1.0 mg/mL insulin solutions in saline (1.0 ml aliquot), stored at −20° C. until use. The dosing solutions were prepared within 2 hours of thawing of the stock solution, using 25 Hg of insulin plus 0, 0.744, 7.44, 37.2, 74.4, 186 or 372 μg of EDTA dissolved in 300 HL of PBS.

### Insulin-EDTA Composition Administration By Intratracheal Instillation

The rats were lightly anesthetized with inhaled 3.0-5.0% Isoflurane (Abbott Laboratories) mixed with oxygen for approximately 5 minutes in a plexiglass anesthesia chamber. The rat was then hung vertically by its upper incisors on a rubber band that was stretched between two burette stands. Approaching the suspended animal from the back, a 3 ml syringe fitted with a gavage needle (Popper & Sons Inc.; 18×3″ W2-¼ mm ball, New Hyde Park, N.Y. 11040) was inserted into the mouth of the rat down the trachea to just above the main carina. When inserting the gavage needle into the trachea, proper insertion can be detected by feeling for the roughness of the cartilage rings under the skin of the throat using the ball of the gavage needle. In contrast, the esophagus is smooth, and should be avoided when dosing. The tip should be positioned just above the main carina by first feeling for the resistance of the main bifurcation of the bronchi, and then by sliding the gavage needle back about 5 mm before dosing. The dose was administered into the lungs utilizing this method, and then the gavage needle was removed. The animals were held in that position for approximately 15 seconds after dosing. The animal was then placed back in its cage and allowed to recover from the anesthesia on its own.

### Sample Collection

Surgically cannulated rats, fitted with a catheter in the jugular vein as described above, were received from the animal supplier. The catheter was held in place within a nape pocket for animal shipment and it was easily exteriorized using a two-person technique in un-anesthetized animals (as per Hilltop instructions). One person gently restrained the rat with both gloved hands against the surface of a lab bench or table. The dorsal surface of the neck between the shoulder blades was exposed so that the second person could gently pry open the retaining nylon suture or metal clip at the nape. The end of the catheter was drawn out of this opening to an approximate 5-cm length, and the metal plug removed from the catheter lumen. The catheter was fitted with a blunt-end metal needle (23Gauge). A 3-mL syringe was fitted into the needle, and the polyvinyl pyrrolidone (PVP) solution present in the catheter lumen was removed by drawing back on the syringe plunger until blood fills the lumen. The PVP and syringe were discarded and replaced with a 1- to 3-mL syringe for blood sample collection. After the sample was collected, an equivalent volume of saline was administered to the rat to flush the blood out of the catheter lumen. The catheter lumen was then filled with heparinized saline (1 to 10 U heparin per mL), and the needle plugged with a cotton applicator tip soaked in heparinized saline. For each sampling time point (Pre-dose and 15, 30, 60, 120, 240 and 360 minutes post-dose), the heparinized saline was removed using a syringe and discarded prior to blood sample collection.

Blood samples of approximately 0.5 ml were collected from the jugular vein catheter into EDTA containing tubes at predose (2 to 0.25 hours prior to dosing), 15, 30, 60, 120, 240 and 360 minutes post-dose. Plasma was separated by centrifugation for 10 minutes at 13,000 rpm in a Baxter Biofuge 13 centrifuge (Baxter, Model Number 3637, Serial Number 187734), and stored frozen at −20° C. until shipped to the sample analysis site on dry ice.

### Sample Analysis

The Vanderbilt Hormone Assay Core Group analyzed plasma samples for insulin levels using immunoassay procedures. Samples were shipped frozen on dry ice to Vanderbilt—Hormone Assay Core Nashville, Tenn.

### Example 1

### The Effect of EDTA on Insulin Bioavailability

To investigate the effect of EDTA on insulin bioavailability, seven test samples containing various amounts of EDTA were prepared. Each test sample contained 25 μg of insulin and 0, 0.744, 7.44, 37.2, 74.4, 186 or 372 μg of EDTA dissolved in 300 μL of PBS. The test samples were administered to rats as described above, blood samples were collected at various time points after dosing, and the amount of insulin in the samples were determined.

Pharmacokinetic analysis of each sample was performed to determine parameters such as the maximum plasma concentration (C_{MAX}), time to maximum plasma concentration (T_{MAX}), area under the plasma concentration vs. time curve (AUC), and apparent elimination half-life (t/2). Analyses were performed using WinNonlin Professional 2.0 (Scientific Consulting, APEX, NC) validated computer program or equivalent.

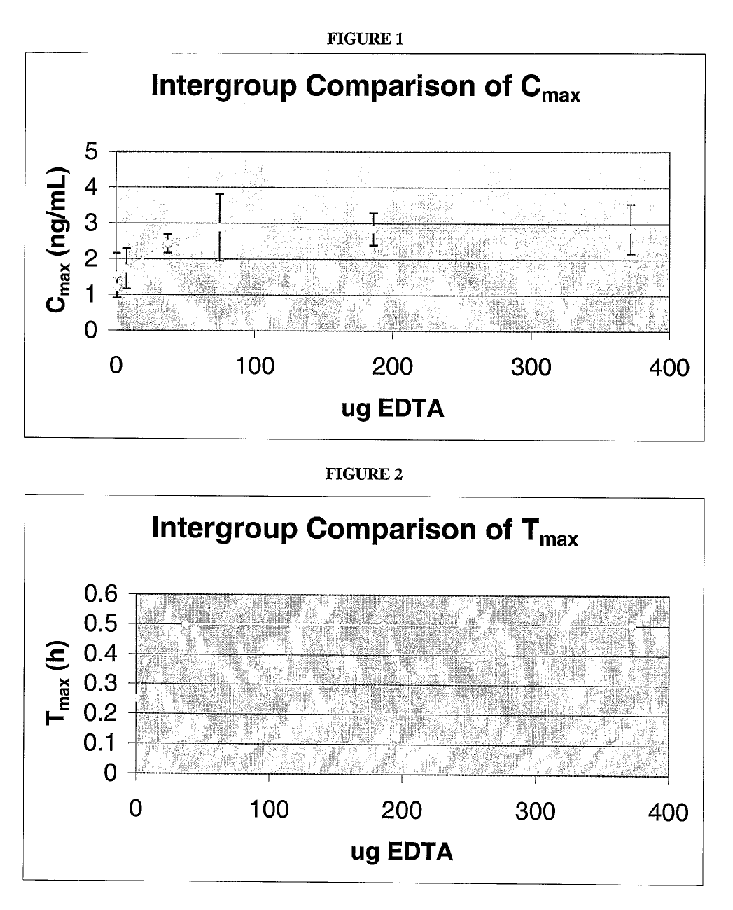

As shown in FIG. 1, the maximum plasma concentration (C_{MAX}) of insulin increases from about 1 ng/ml with no EDTA to about 3 ng/ml with 74.4 μg of EDTA. The C_{MAX} of insulin appears to plateau at this level of EDTA, as further addition of EDTA did not increase the plasma concentration of insulin. The time to maximum plasma insulin concentration (FIG. 2) showed a similar plateau effect. With no EDTA in the sample solution, T_{MAX} for insulin was about 0.2 hours. This increased to about 0.5 hours when 37.2 μg of EDTA were present in the solution. Higher amounts of EDTA did not increase or decrease the T_{MAX} significantly from about 0.5 hours.

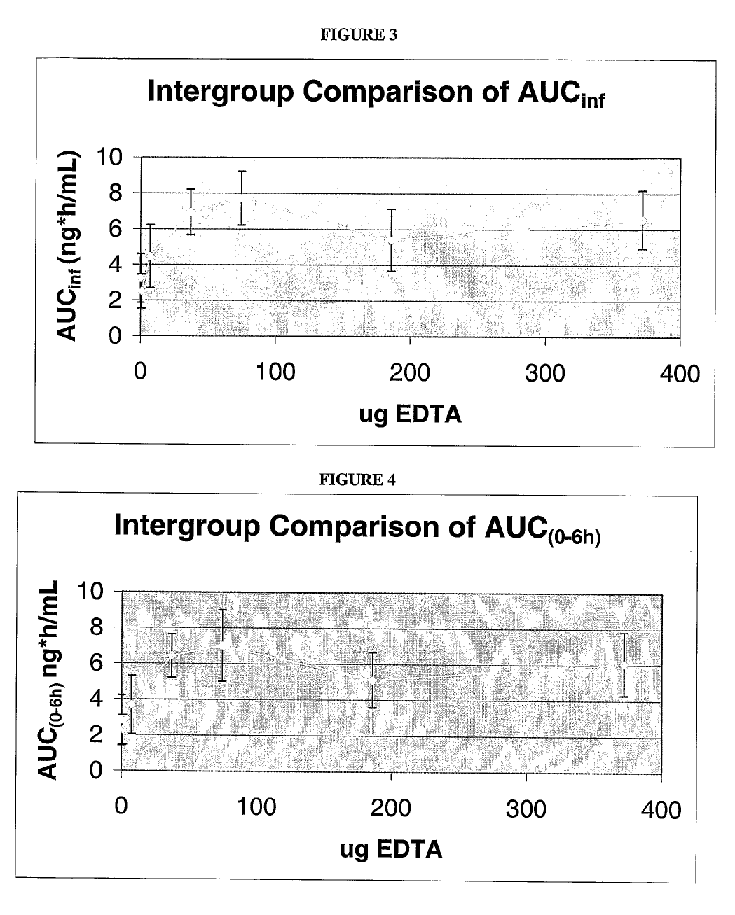

As shown in FIGS. 3 and 4, the area under the plasma concentration vs. time curve (AUC) for insulin also increased with the addition of EDTA in the composition. With no EDTA the AUC of insulin was approximately 2 (ng*h)/ml. The AUC increased with increasing amounts of EDTA to about 7.2 (ng*h)/ml when there were 74.4 μg of EDTA in the dosing solution. The effect of added EDTA on the AUC from 0 to 6 hours post dosing (FIG. 4) was similar to the effect on the AUC from 0 to infinity (FIG. 3).

The apparent elimination half-life of insulin (FIG. 5), on the other hand, did not change significantly with varying amounts of EDTA. Therefore, EDTA did not affect the ability of the body to clear insulin from blood.

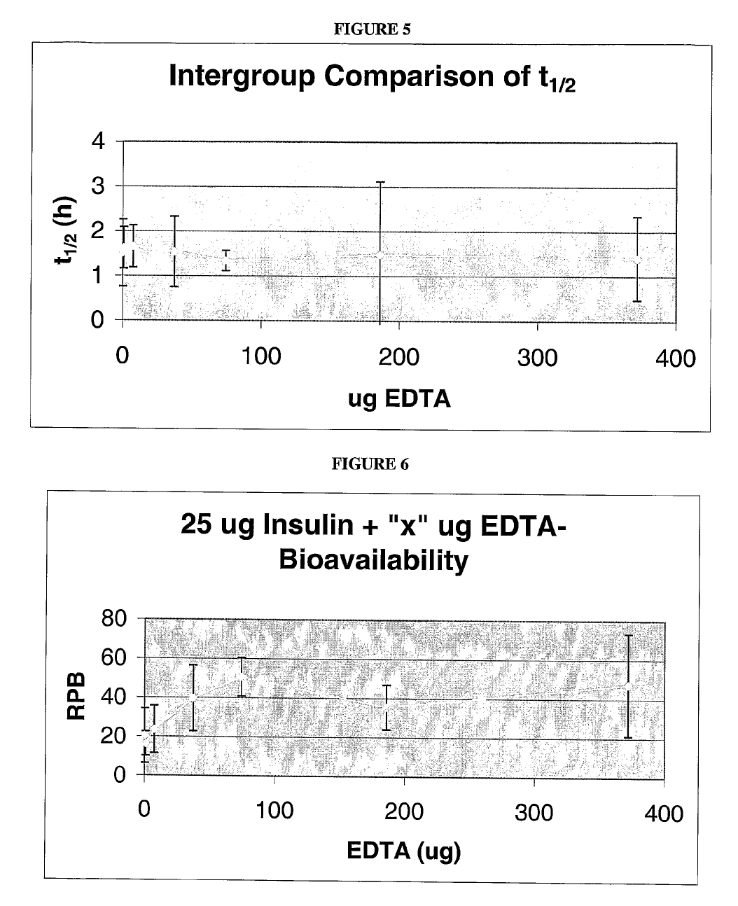

FIG. 6 shows a comparison of the bioavailability of insulin in each of the seven separate test samples. The bioavailability (% F) was first determined, then the relative pulmonary bioavailability was derived from a comparison to the bioavailability of a subcutaneous composition. As shown in FIG. 6, the relative pulmonary bioavailability was significantly enhanced by the addition of EDTA. Thus, the peak bioavailability under these administration conditions was about 50% of that of a subcutaneous injection, which is a great improvement from the 20% without EDTA.

These results thus indicate that EDTA can be used to effectively increase the bioavailability, C_{MAX}, T_{MAX} and AUC of insulin compositions.

Variations and equivalents of this example will be apparent to those of skill in the art in light of the present disclosure, the drawings and the claims herein.

All articles, books, patents and other publications referenced herein are hereby incorporated by reference in their entirety.

We claim:

1. A pharmaceutical composition suitable for pulmonary delivery, comprising:
insulin; and
EDTA;
wherein the presence of said EDTA is effective to increase the relative pulmonary bioavailability of the insulin compared to the relative pulmonary bioavailability exhibited by a composition having the same components but absent said EDTA, and
wherein the composition is in dry powder form.

2. The pharmaceutical composition of claim 1, wherein the EDTA is a sodium salt of ethylene diaminetetraacetic acid.

3. The pharmaceutical composition of claim 1, comprising from about 5 to about 90 solid weight percent of said EDTA.

4. The pharmaceutical composition of claim 1, comprising from about 10 to about 85 solid weight percent of said EDTA.

5. The pharmaceutical composition of claim 1, comprising from about 30 to about 70 solid weight percent of said EDTA.

6. The pharmaceutical composition of claim 1, comprising from about 50 to about 70 solid weight percent of said EDTA.

7. The pharmaceutical composition of claim 1, wherein particles of the powder have a mass median diameter (MMD) from about 1-5 microns.

8. The pharmaceutical composition of claim 1, wherein particles of the powder have a mass median aerodynamic diameter (MMAD) of less than about 10 microns.

9. The pharmaceutical composition of claim 1, wherein particles of the powder have an MMAD of less than about 5 microns.

10. The pharmaceutical composition of claim 1, wherein particles of the powder have an MMAD of less than about 3.5 microns.

11. The pharmaceutical composition of claim 1, which, when administered by inhalation, results in a relative pulmonary bioavailability that is at least about 3 fold of the relative pulmonary bioavailability of a composition having the same components and absent said EDTA.

12. The pharmaceutical composition of claim 1, which, when administered by inhalation, results in a relative pulmonary bioavailability that is at least about 2 fold of the relative pulmonary bioavailability of a composition having the same components and absent said EDTA.

13. The pharmaceutical composition of claim 1, which, when administered by inhalation, results in a relative pulmonary bioavailability that is at least about 1.5 fold of the relative pulmonary bioavailability of a composition having the same components and absent said EDTA.

14. The pharmaceutical composition of claim 1, which, when administered by inhalation, results in a relative pulmonary bioavailability that is at least about 1.2 fold of the relative pulmonary bioavailability of a composition having the same components and absent said EDTA.

15. A method of treating or ameliorating diabetes or a related condition in a mammal, which method comprises administering by inhalation a pharmacologically effective amount of the composition of claim 1.

16. The method of claim 15, wherein the EDTA is a sodium salt of EDTA.

17. The method of claim 15, wherein the composition comprises from about 5 to about 90 solid weight percent of said EDTA.

18. The method of claim 15, wherein the composition comprises from about 10 to about 85 solid weight percent of said EDTA.

19. The method of claim 15, wherein the composition comprises from about 30 to about 75 solid weight percent of said EDTA.

20. The method of claim 15, wherein the composition comprises from about 50 to about 70 solid weight percent of said EDTA.

21. The method of claim 15, wherein particles of the powder have an MMD from about 1-5 microns.

22. The method of claim 15, wherein particles of the powder have an MMAD of less than about 10 microns.

23. The method of claim 15, wherein particles of the powder have an MMAD of less than about 5 microns.

24. The method of claim 15, wherein particles of the powder have an MMAD of less than about 3.5 microns.

25. The method of claim 15, wherein the inhalation results in a relative pulmonary bioavailability that is at least about 3 fold of the relative pulmonary bioavailability of a composition having the same components and absent said EDTA.

26. The method of claim 15, wherein the inhalation results in a relative pulmonary bioavailability that is at least about 2 fold of the relative pulmonary bioavailability of a composition having the same components and absent said EDTA.

27. The method of claim 15, wherein the inhalation results in a relative pulmonary bioavailability that is at least about 1.5 fold of the relative pulmonary bioavailability of a composition having the same components and absent said EDTA.

28. The method of claim 15, wherein the inhalation results in a relative pulmonary bioavailability that is at least about 1.2 fold of the relative pulmonary bioavailability of a composition having the same components and absent said EDTA.

29. The method of claim 15, wherein the composition is administered using a dry powder inhaler.

30. The method of claim 15, wherein the composition is administered using an MDI.

31. The method of claim 15, wherein said pharmacologically effective amount is administered in a plurality of unit dosages.

32. A method of improving the pulmonary bioavailability of an insulin composition, comprising adding EDTA to an insulin composition suitable for pulmonary delivery, wherein the composition is in dry powder form.

33. The method of claim 32, wherein the EDTA is a sodium salt of EDTA.

34. The method of claim 32, wherein the composition comprises from about 5 to about 90 solid weight percent of said EDTA.

35. The method of claim 32, wherein the composition comprises from about 10 to about 85 solid weight percent of said EDTA.

36. The method of claim 32, wherein the composition comprises from about 30 to about 75 solid weight percent of said EDTA.

37. The method of claim 32, wherein the composition comprises from about 50 to about 70 solid weight percent of said EDTA.

38. The method of claim 32, wherein particles of the powder have an MMD from about 1-5 microns.

39. The method of claim 32, wherein particles of the powder have an MMAD of less than about 10 microns.

40. The method of claim 32, wherein particles of the powder have an MMAD of less than about 5 microns.

41. The method of claim 32, wherein particles of the powder have an MMAD of less than about 3.5 microns.

42. The method of claim 32, wherein the improvement results in a relative pulmonary bioavailability that is at least about 3 fold of the relative pulmonary bioavailability of a composition having the same components and absent said EDTA.

43. The method of claim 32, wherein the improvement results in a relative pulmonary bioavailability that is at least about 2 fold of the relative pulmonary bioavailability of a composition having the same components and absent said EDTA.

44. The method of claim 32, wherein the improvement results in a relative pulmonary bioavailability that is at least about 1.5 fold of the relative pulmonary bioavailability of a composition having the same components and absent said EDTA.

45. The method of claim 32, wherein the improvement results in a relative pulmonary bioavailability that is at least about 1.2 fold of the relative pulmonary bioavailability of a composition having the same components and absent said EDTA.

46. The method of claim 32, wherein the composition is administered using a dry powder inhaler.

47. The method of claim 32, wherein the composition is administered using an MDI.

48. The method of claim 32, wherein said pharmacologically effective amount is administered in a plurality of unit dosages.
